(19) United States
(12) Patent Application Publication
(10) Pub. No.: US 2017/0027482 A1
(43) Pub. Date: Feb. 2, 2017

(54) WIRELESS COLORIMETRIC SENSOR

(71) Applicant: SPECTROPHON LTD, Rehovot (IL)

(72) Inventors: Gleb ZILBERSTEIN, Rehovot (IL); Shmuel BUKSHPAN, Ramat Hasharon (IL)

(21) Appl. No.: 15/303,765

(22) PCT Filed: Apr. 2, 2015
(86) PCT No.: PCT/IL2015/050360

§ 371 Date: Oct. 13, 2016

Related U.S. Application Data
(60) Provisional application No. 61/978,913, filed on Apr. 13, 2014.

(51) Int. Cl. A61B 5/145; A61B 5/00; G01N 33/543; G01N 33/497; G01N 33/487

(52) U.S. Cl. CPC A61B 5/14517; G01N 33/497; G01N 33/4972; G01N 33/48792; G01N 33/54386; A61B 5/681; A61B 5/0022

(57) ABSTRACT

A wireless colorimetric sensor for detecting, quantifying, or both detecting and quantifying, at least one analyte in a sample, the sensor including a light transparent chemochromic layer applied onto the photoactive surface of an image sensor, or attached thereto via an optic coupler, wherein the chemochromic layer comprises a chemochromic material that is optically altered when contacted with the analyte. A method for detecting at least one analyte with a colorimetric sensor.

### FIELD OF THE INVENTION

Embodiments of the invention are directed to a colorimetric sensor for detection of volatile, volatilized or liquid chemical analytes, based on colorimetric sensing by a chemochromic layer deposited on the photoactive surface of a CMOS or CCD image sensor, or any other appropriate sensor, coupled to a radio chip by Wi-Fi, Bluetooth, or any other appropriate means. Further, the invention is directed to the combination of a colorimetric variable optical filter layer deposited on the photoactive surface of an image sensor, gathering optical information, wireless transmission of the optical information to a processing hub and the application of processing software for the detection and, possibly, quantification of an analyte.

### BACKGROUND OF THE INVENTION

Methods of colorimetric detection and sensing of specific chemical analytes are widely known in the art. Such methods are based on a process that induces a reversible or an irreversible change in the optical properties (color, transparency, light scattering intensity, etc.), of specific active compounds when exposed to a chemical analyte. Many natural compounds are known to exhibit chromism, and many artificial compounds with specific chromism have been synthesized and utilized as chromic sensors.

Detection and quantification of the changes in the optical parameters of a chemochromic material allows for the measurement of the quantity of a specific analyte to which the chemochromic material was exposed. Mostly, each analyte is detected by a certain chromic sensor and therefore, such detection methods tend to be highly specific.

Chromic sensing has been widely utilized in to identify the presence or level of specific analytes in liquid solutions or and in gas phase mixtures.

Known in the art are also attachable or integrated electronic sensors that can be attached to or integrated in various electronic devices, including mobile electronic devices, such as cell phones, wherein the data regarding the analytes is collected and analyzed by the attachable electronic sensor. Examples of such attachable/integrated sensors are found, for example, in US 2009/0325639, US 2004/0081582 and JP 2005/5086405.

iBreath Alcohol Breathalyzer (iBAB) is an example of a personal alcohol measuring module that connects to an iphone. The iBAB connects to the iphone for power and for possible

transmission of the results to third parties; however, the detection and analysis of the alcohol content if performed in the iBAB itself, not on the iphone.

Other methods of utilizing chemochromic materials coated on optical fibers or LED based sensors are known.

Numerous patents utilize digital cameras of mobile electronic devices such as smartphones, tablets, etc., for recording and analyzing reflection from colorimetric sensors, such as strips, microfluidic chips, custom designed sampling chips and arrays for analysis of liquid or gaseous samples. Thus, there is a need in the art for an inexpensive, low cost, highly sensitive, wireless sensor for personal use that may be used to detect various types of analytes easily, using widespread technology.

### SUMMARY OF THE INVENTION

Embodiments of the invention are directed to a wireless colorimetric sensor for detecting, quantifying or both detecting and quantifying, at least one analyte in a sample, the sensor comprising a light transparent chemochromic layer applied onto the photoactive surface of an image sensor, or attached thereto via an optic coupler, wherein the chemochromic layer comprises a chemochromic material that is optically altered when contacted with the analyte.

Further embodiments of the invention are directed to a method for detecting at least one analyte with a colorimetric sensor comprising an image sensor, wherein a chemochromic layer comprising a chemochromic material is applied onto the photoactive surface of the image sensor, or attached thereto via an optic coupler, the method comprising:

exposing at least part of the chemochromic material to an analyte;

transmitting light through the at least part of the chemochromic layer;

recording a digital optical pattern of light transmitted through at least part of the chemochromic layer;

transmitting the digital optical pattern to an image processing device; and

comparing the digital optical pattern with a digital optical reference pattern.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 presents a schematic diagram of an exemplary chemochromic sensor according to an embodiment of the invention;

FIG. 2 presents a top view of a chemochromic layer coated on the photoactive surface of an image sensor, such as a CCD or CMOS sensor;

FIG. 3 presents a top view of a two-element matrix having two different chemochromic regions coated on the photoactive surface;

FIG. 4 presents a top view of a multi-region matrix chemochromic layer coated on a photoactive surface;

FIG. 5 presents an embodiment of a chemochromic sensor comprising an optical coupler;

FIG. 6 presents an embodiment of a chemochromic sensor with a capillary pump for liquid sampling.

FIG. 7 presents an embodiment of a sensor in which a chemochromic layer was applied, and an external UV source;

FIG. 8 presents an embodiment of constant volume sampling devices.

FIG. 9A presents an embodiment of a chemochromic sensor that may be reset to its original optical state by IR radiation after being exposed to an analyte;

FIG. 9B presents an embodiment of a chemochromic sensor, which may be reset to its original optical state by a heating element after being exposed to an analyte;

FIG. 9C presents an embodiment of a chemochromic sensor, which may be reset to its original optical state by ultrasound after being exposed to an analyte.

### DETAILED DESCRIPTION OF THE INVENTION

Some embodiments of the invention are directed to a wireless colorimetric sensor for detecting, and possibly quantifying, as least one analyte in a sample, the sensor comprising a light transparent chemochromic layer, which comprises a chemochromic material that is optically altered when contacted with the analyte and which is applied onto the photoactive surface of an image sensor.

According to some embodiments, the image sensor is a CMOS or CCD sensor. According to some embodiments, the colorimetric sensor is miniature. According to some embodiments, the image sensor has a length of about 5-10 mm. According to some embodiments, the image sensor has a length of about 10-15 mm. According to some embodiments, the image sensor has a length of about 15-50 mm. According to some embodiments, the image sensor has a length of about 50-100 mm. According to some embodiments, the image sensor has a length of at least 5 mm.

According to some embodiments, the colorimetric sensor is ultra-low power consuming. According to some embodiments, the colorimetric sensor includes means by which recorded optical data may be transferred to any appropriate type of processing device. According to some embodiments, the colorimetric sensor is remote radio communication enabled. According to some embodiments, the optical data is transferred by Wi-Fi, Bluetooth or near field communication (NFC). According to some embodiments, the processing device is a smartphone, tablet, computers or any type of digital hub comprising graphic processing software.

Certain embodiments of the invention are directed to a method for detecting at least one analyte with a colorimetric sensor comprising an image sensor, wherein a chemochromic layer comprising a chemochromic material is applied onto the photoactive surface of the image sensor, or attached thereto via an optic coupler, the method comprising

exposing the at least part of chemochromic material to an analyte;

transmitting light through at least part of the chemochromic layer;

recording a digital optical pattern of light transmitted through at least part of the chemochromic layer;

transmitting the digital optical pattern to an image processing device; and

comparing the digital optical pattern with a digital optical reference pattern.

According to some embodiments, the digital optical pattern is transmitted wirelessly. According to some embodiments, the digital optical pattern is transmitted by Wi-Fi or Bluetooth. According to some embodiments, the digital optical pattern is compared with the digital optical reference pattern using any appropriate type of digital processing, such as image analysis software. In this respect it is noted that, unless specifically stated otherwise, any reference herein to “image analysis software” includes any appropriate type of digital processing.

According to some embodiments, the digital optical reference pattern is obtained by transmitting light through at least part of the chemochromic layer before the chemochromic material is exposed to the analyte. According to further embodiments, the digital optical reference pattern is obtained by transmitting light through at least part of the chemochromic layer of any appropriate sensor, wherein the digital optical reference pattern is stored in memory for further use. According to some embodiments, the digital optical reference pattern is obtained from regions of the chemochromic layer that do not contain chemochromic material. According to some embodiments, the digital optical reference pattern or patterns stored in memory include quantitative data, such that the comparison of the obtained digital optical pattern thereto may provide quantitative data regarding the amount of the analyte in the measured sample.

According to some embodiments, the chemochromic layer comprises a solid layer comprising a chemochromic compound (or material), which is specific to a certain type of analyte. According to some embodiments, the chemochromic compound is uniformly dispersed in a transparent media. According to some embodiments, the chemochromic containing media is applied to the photoactive surface of an image sensor by any appropriate means, such as printing, polymerization, ink jet, adhesion, mechanical attachment and the like. According to some embodiments, the chemochromic layer covers the entire area of the photoactive surface. According to some embodiments the chemochromic layer covers only part of the photoactive surface of the image sensor.

According to some embodiments of the invention, the chemochromic layer comprises at least two different chemochromic materials, such that at least two analytes may be simultaneously detected. According to some embodiments, each of the chemochromic materials is found in a predefined area. According to other embodiments chemochromic layer comprises a matrix of subsections, wherein each subsection comprises a different chemochromic material, thus enabling the simultaneous detection of multiple analytes.

According to some embodiments, a predefined area on the chemochromic layer, such as one of the matrix elements, comprises only the transparent media, without the chemochromic compound (or material. It is noted that the terms “chemochromic material” and “chemochromic compound” as used herein may be interchanged). Such an area, free from chemochromic material, may be employed for calibration and used as a reference, referred to herein also as a digital optical reference pattern.

In another embodiment of the invention, the sensor comprises an optical coupler attached to the photoactive face of an image sensor and a chemochromic layer deposited on the surface of the optical coupler, such that the coupler is placed between the image sensor and the chemochromic layer. According to some embodiments, the coupler comprised a transparent media, such as, a polymer layer, polymer disc, transparent glass disk, optical collimator or any optically transparent matter. According to such an embodiment, an optical pattern recorded after the exposure of the sensor to the analyte or mixture of analytes may be compared to a separate reference. Any number of optical couplers may be attached to a single image sensor, wherein a chemochromic layer is placed at the distal end of each optic coupler. The chemochromic layers on the distal ends of the optical couplers may be identical to one another or different from one another.

In such an embodiment, the optical coupler may be a flexible light guide, an optical fiber or bundle of optical fibers attached to the photoactive surface of the image sensor on one end and coated by a chemochromic layer on the opposite surface of the bundle, each bundle detecting specific analytes at same or different sites and distances from the image sensor.

The use of optical couplers may aid in preparing a flat device, such as an armband, bracelet or cloth, that contain a colorimetric sensor. Further, the use of optical couplers enables the preparation of a colorimetric sensor for measuring more than one analyte, wherein each analyte is measured in the appropriate region. For instance, two optical couplers may be connected to photoactive face of an image sensor, wherein the distal end of the first optical coupler includes a chemochromic layer intended to detect alcohol and distal end of the second optical includes a chemochromic layer intended to detect sweat odor. Thus, the distal end of the first optical coupler may be placed near the mouth, while the distal end of the second optical coupler may be placed near the armpit.

According to some embodiments, the colorimetric sensor does not include an optical lens. According to other embodiments, the colorimetric sensor includes an optical lens. When the colorimetric sensor includes an optical lens, at least part of the lens may be covered with at least part of the chemochromic layer. According to some embodiments, when the colorimetric sensor includes an optical coupler, the optical lens is positioned at the distal end of the optical coupler.

According to some embodiments, the chemochromic material reacts with the analyte by any known mechanism, such as changes in color, transparency, light scattering intensity, fluorescence with external excitation, chemoluminescence, thermoluminescence, phosphorescence, plasmon based light scattering, or any combination thereof.

Unless specifically stated otherwise, exposing the sensor to the analyte and/or the sample is interchangeable with contacting the chemochromic layer/material with the analyte/sample.

According to some embodiments, the analyte is in a form of gas, aerosol, droplets, spray, plasma, flame, airborne liquid or solid particles or any other form that may be carried in the surrounding atmosphere. According to some embodiments, a volatile analyte is contained in a predetermined volume compartment. According to further embodiments, the analyte may be in any type of medium, such as gas, liquid, solid, gel, cream, or the like.

The chemochromic layer may be exposed to the analyte by spreading a sample containing the analyte on the chemochromic layer, using, e.g., a swab, or the like, a capillary pump dipped in a vessel containing the analyte, a spray, aerosol, gas stream, brush, or by any other appropriate means. According to some embodiments, the colorimetric sensor is exposed to a predetermined volume of the sample, such that a quantitative assessment of the concentration of the analyte in the sample may be determined. According to some embodiments, the predetermined volume is set be a capillary pump, a pipette, a syringe, a balloon, or any other appropriate means.

As detailed above, a digital optical pattern must be obtained in order to assess the type and/or amount of the analyte in the sample. In order to obtain the digital optical pattern, the chemochromic layer may be illuminated or radiated by any appropriate electromagnetic radiation, including visible light, UV, IR, X-rays, microwaves, etc. Accordingly, any reference to “light” or the like herein, should be understood to include any type of electromagnetic radiation, as well as electroluminescence, chemoluminescence, fluorescence, and phosphorescence, induced inside or outside of the chemochromic layer of the sensor. According to further embodiments, the illumination or radiation means are ambient light or artificial light like a white LED or any other means of illumination, such that they are a physical part of a monolithic sensor or any part of an enclosure or device the colorimetric sensor is incorporated into.

According to some embodiments, the light source may be internal, i.e., part of the colorimetric sensor, or external (natural or artificial). The light source may be sunlight, filtered light, white light, laser, diode light or any other appropriate light or radiation source.

According to some embodiments, the chemochromic material is considered to be any type of material that changes are induced by exposure to the analyte, such that the changes may be detected using light/radiation.

According to some embodiments, the chemochromic is transparent or translucent, such that the electromagnetic radiation probes the whole active volume/thickness of the chemochromic layer deposited on the photoactive surface of the image sensor, such that the electromagnetic radiation passes through the entire thickness of the chemochromic layer, operating by transmission and not reflection and/or refraction. The use of transmission, rather than refraction/reflection, enhances the sensitivity of the measurement. Such enhancement allows the detection of miniscule amounts of the analyte, which could not have been detected using reflection and/or refraction methods.

In order to implement transmission, the chemochromic layer of the sensor is prepared such that it is transparent/transluscent. Further, according to some embodiments, the chemochromic material is dispersed throughout the entire thickness of the chemochromic layer and the optical changes caused to the chemochromic material due to the contact thereof with the analyte occur throughout that thickness. According to such embodiments, the chemochromic layer is prepared such that it is porous, thereby allowing the penetration of the measured sample to the entire thickness thereof. According to some embodiments, the deposited chemochromic layer has a thickness of at least about 10 nm. According to further embodiments, the chemochromic layer has a thickness of 10 microns. According to further embodiments, the chemochromic layer has a thickness of about 100 microns. According to some embodiments, the chemochromic layer has a thickness of about 1 mm. According to some embodiments, the chemochromic layer has a thickness of about 1 cm.

According to some embodiments, the active materials used to detect the analyte, are not sensitive to any materials other than the analyte itself, including any type of humidity, such as water, water vapor, water droplets, water aerosol and the like. According to such embodiments, the chemochromic layer is not sensitive to humidity and therefore, may be used under any surrounding conditions, including humid conditions.

The colorimetric sensors detailed herein may be used for any necessary means, such as medical testing, law enforcement, environmental testing, security and the like. According to further embodiments, the method is considered to be social method for use by any individual or group of laymen desiring to detect any condition, such as, without limitation, alcohol consumption, bad breath or body odor.

According to some embodiments the colorimetric sensor is designed to be disposable. According to further embodiments, the sensor is designed for multiple use. For multiple use the sensor is designed with proper chemochromic layer and microstructure enabling the chemochromic material to reset. According to some embodiments, the reset of the chemochromic material is fast and traces of the analyte are removed therefrom. The colorimetric sensor may contain a weakly binding chemochromic compound allowing self reset in short time intervals. According to other embodiments, the colorimetric sensor may be actively reset by any appropriate means, such as heating (using hot air, IR, heating elements and the like).

According to some embodiments, the acquisition of the reference data is performed in the same surrounding conditions as the sampling measurements, such that the comparison between the digital optical pattern and the digital optical reference pattern is such that the differences between the two may be attributed practically completely to the exposure of the chemochromic material to the analyte. The surrounding conditions include the lighting conditions, the white balance parameters, the data acquisition time etc.

According to the invention the optical change occurring in the chemochromic layer of the colorimetric sensor when exposed to the analyte should be in the sensitivity range of the image sensor under the ambient or artificial illumination. According to another embodiment, the optical change recorded by the sensor exposed to the analyte may be based on the change in any appropriate electromagnetic radiation, e.g., visible light, UV fluorescence, IR, thermoluminescence, chemoluminescence, bioluminescence, phosphorescence, long glow time phosphorescence, x-rays phosphorescence, electroluminescence, fluorescent materials with long period of post luminescence, etc., or any combination thereof.

According to some embodiments, the chemochromic layer comprises only the chemochromic material. According to further embodiments, the chemochromic layer includes additional substances. According to further embodiments, the chemochromic layer further includes a transparent or translucent substance. According to some embodiments, the additional substance is porous. According to some embodiments, the additional substance includes a media containing a silica gel, polyacrylamide gel, sol gel, polyvinyl alcohol or any combination thereof. According to some embodiments, the additional substances may include a light dispersing material to homogenize the optical response throughout the volume of the chemochromic layer to the transmitted light According to some embodiments the chemochromic layer contains luminescent or phosphorescent compounds for light generation in the volume of the chemochromic layer, such that external light is not required.

According to such embodiments, the chemochromic material is integrated with the additional components of the chemochromic layer by any appropriate means, such as a solution, suspension, dispersion or the like.

According to some embodiments, the chemochromic layer is applied onto the photoactive surface by any appropriate means, including laminating, spraying, coating or casting. According to some embodiments, as detailed herein, the chemochromic layer is applied to the distal end of an optic coupler. Such application may also be by any appropriate means, including laminating, spraying, coating or casting.

According to other embodiments, the chemochromic layer is a membrane, patch, label, diaphragm, film attached to the photoactive surface of the sensor by any appropriate means, such as any appropriate type of adhesive.

The thickness of the chemochromic layer as well as the amount and concentration of the chemochromic material, depend on the specific type of the sample, the amount of analyte to be detected, and the specific chemochromic material used. According to some embodiments, the thickness of the chemochromic layer is between 10-100 micron. According to some embodiments, the thickness of the chemochromic layer is between 10-20 micron. According to some embodiments, the thickness of the chemochromic layer is between 20-30 micron. According to some embodiments, the thickness of the chemochromic layer is between 30-40 micron. According to some embodiments, the thickness of the chemochromic layer is between 40-50 micron. According to some embodiments, the thickness of the chemochromic layer is between 50-60 micron. According to some embodiments, the thickness of the chemochromic layer is between 60-70 micron. According to some embodiments, the thickness of the chemochromic layer is between 70-80 micron. According to some embodiments, the thickness of the chemochromic layer is between 80-90 micron. According to some embodiments, the thickness of the chemochromic layer is between 90-100 micron. According to some embodiments, the thickness of the chemochromic layer is above 100 micron. According to some embodiments, the thickness of the chemochromic layer is between 100-200 micron. According to some embodiments, the thickness of the chemochromic layer is between about 1 mm-1 cm. According to some embodiments, the concentration of the chemochromic active compound in the layer is 10 nM-1M. According to some embodiments, the concentration of the chemochromic material is at least 10 nM. According to some embodiments, the concentration of the chemochromic material is not higher than 1M.

According to some embodiments, the sample is a gas. According to other embodiments the sample is a liquid volatilized by means of evaporation, aerosol formation or nebulization. According to another embodiment the sample is a gaseous suspension of solid particles obtained, for example, by thermal volatilization. According to further embodiments, the sample is a liquid, solid, gel, plasma, flame, cream and the like.

According to some embodiments, the chemochromic layer of the sensor is exposed to exhaled breath, i.e., the detected analyte is a substance found in the breath of the user. The necessary amount of chemochromic material and thickness of the chemochromic layer are correlated with the specific analyte concentration range, the volume of the sampled gas, breath, or liquid and by the required sampling time.

According to some embodiments, the sensor is exposed to a single breath. According to further embodiments, the sensor is exposed to at least two breaths or to a certain volume which is determined by one skilled in the art.

According to some embodiments, the volume of the sample containing the analyte is predefined, such that a quantitative analysis of the amount of analyte may be performed. Such a quantitative analysis may include the use of means such as a calibrated volume balloon for gas or a pipette for liquids, to ensure that an exact volume of the sample is measured. According to some embodiments, such a quantitative analysis may be used for medical means.

According to some embodiments, a calibration curve is prepared testing samples with a known amount of the analyte. Such a calibration curve may be stored for future use in determining the amount of the analyte in samples wherein the concentration of the analyte is unknown. In another embodiment a calibrated reference pattern may be stored. According to some embodiments, a calibration curve may be prepared by measuring a fixed component in the sample, such as nitrogen (or any other inert gas) in breath. According to some embodiments, a calibration curve may be prepared for each individual using the system. An individual calibration curve may be prepared by averaging a multiple number of tests performed on that specific individual.

According to other embodiments, the image analysis software provides a qualitative analysis. According to some embodiments, the image analysis software provides results relating to the presence or absence of the analyte according to a predefined threshold relating to the optical changes in the chemochromic material. According to further embodiments, the image analysis software provides a result that is positioned, graphically and/or numerically, on a color bar and/or in a range list, which includes several ranges relating to the amount or presence of the analyte. According to some embodiments the ranges include a “not identified” range, “low amount” range and “high amount” range, relating to the presence of the analyte in the tested sample (or any similarly noted ranges). According to further embodiments, the ranges include a “not identified” range and an “identified” range (or any similarly noted ranges).

According to some embodiments, the chemochromic layer is in liquid form contained in a properly designed optically transparent vessel attached to the photoactive surface of the image sensor.

According to some embodiments, the sample is a gas. According to some embodiments, the sample is a liquid. According to some embodiments, the sample is a solid.

According to some embodiments, the reaction of the chemochromic material in the chemochromic layer is reversible, i.e., the colorimetric sensor returns to the optical state it was in before exposure to the analyte, within a certain period of time after exposure. According to some embodiments, when the chemochromic material is reversible, the colorimetric sensor may be reused after a predetermined time, disregarding the prior use thereof. According to another embodiment a fast reset colorimetric sensor can continuously monitor the level of a specific analyte. According to other embodiments, the fast reset sensor detects time varying or periodic events in which the analyte level changes.

According to some embodiments, the chemochromic material is actively returned to its original optical state, e.g., by heating, mechanical vibration, ultrasound, chemical reaction, microwaves, and the like or any combination thereof. According to some embodiments, the colorimetric sensor should be stored under controlled conditions.

According to other embodiments, the reaction of the chemochromic material is irreversible. Such irreversible chemochromic materials may be used for measuring the accumulation of certain analytes in multiple or long time exposures. According to some embodiments, irreversible chemochromic materials may be used to prepare a graph showing the amount, or relative amount, of the analyte, by comparing the reaction of the chemochromic material at pre-set time points. An example of such a measurement would be the use of the method of the present invention to monitor the amount of specific pollutants in any type of environment, e.g., home, work, public areas, outdoors, etc. The comparison performed by the image analysis software between different times will enables the preparation of a graph demonstrating the amount of accumulated pollutant at every time point that the chemochromic layer was exposed to light or radiation, thus enabling the user to follow the pollutant concentration in the tested area as well as the accumulated effect thereof.

According to some embodiments, the colorimetric sensor is designed to be disposable, i.e., for a single use. According to further embodiments, the colorimetric sensor is designed for multiple use. According to further embodiments the sensor is enclosed in a kit containing a user instruction leaflet. According to some embodiments, the kit comprises a calibration scale, relating to the concentration of the analyte. According to some embodiments, the calibration scale is provided digitally by the image analysis software or any accompanying software. According to some embodiments, the kit comprises a calibrated volume balloon for collecting a specific, predetermined volume of the sample and then exposing the sensor to that specific volume.

According to some embodiments, the software includes a digital optical reference pattern obtained by the sensor that was not exposed to the analyte, under determined light/radiation conditions. According to another embodiment the software includes a digital optical reference pattern of the sensor that was exposed to a calibrated volume and concentration of the analyte, under determined lighting/radiation conditions.

According to further embodiments, the digital optical reference pattern may be downloaded by the user from the internet or incorporated in the kit on a flash memory chip or by any other appropriate means.

According to some embodiments the colorimetric sensor is designed to detect a single analyte.

According to other embodiments, the colorimetric sensor is designed to detect multiple analytes. According to such embodiments, the chemochromic layer deposited on the photoactive surface of the image sensor comprises a matrix of different chemochromic compounds, each of the compounds for the detection of a specific analyte. The area of each chemochromic compound in the chemochromic matrix may be designed to be equal or different in shape and/or size.

According to other embodiments, a fast reset colorimetric sensor is used as a monitoring device and alarm. By continuous sensing the analyte level in the sampled environment and recording fluctuations in the level of the analyte in time, safety exceeding levels are monitored and may be reported by any appropriate means, such as an alarm, digital message to operator or user, and the like.

According to the invention, each colorimetric sensor is designed to detect a certain type or types of analyte, set according to the desired use of the method of the invention. Types of analytes include volatile sulfur compounds, such as H2S or mercaptan, for measuring bad breath, alcohol in breath or ethyl glucuronide in urine, for measuring recent alcohol consumption, acetone in breath or urine, indicating glucose blood levels and fat burning levels, particularly beneficial for diabetics, determination of traces of gluten in food samples, chlorine and metal contaminants like lead, chromium and other metallic elements in water, hydrogen, methane, carbon monoxide, carbon dioxide, benzene, any type of pollutant and various organic compounds, such as formaldehyde, benzoyl peroxide and others.

The chemochromic materials found in the chemochromic layer of the sensor are chosen according to the designation of the specific colorimetric sensor manufactured, i.e., according to the type of analyte/s to be detected. For example, volatile e sulfur compounds, such as H2S and mercaptane, may be detected by potassium thiocyanate and ammonium molybdate, which change from colorless to red upon exposure to such compounds, lead acetate, which changes from colorless to black, 5′5-dithiobis(2-nitrobenzoic acid), which changes from white to yellow, Neocuproine-Cu(II) and/or CuSO4. Such compounds are known to detect about 0.1-1.0 ppm volatile sulfur compounds in gas, e.g., in breath. Sulfur dioxide may be detected using dichloro-bis(diphenylphosphino) and/or methane dipalladium(I), possibly immobilized in a PVC membrane plasticized with o-nitrophenyloctylether. Ethanol may be detected by polydiacetylene/ZnO nanocomposites, acidic solutions of chromium(VI) salt, acidic chromate supported on a silica gel and/or Dye CR-546. Such compounds are known to detect about 10-100 pg ethanol in 100 ml gas, e.g., breath. Acetone may be detected by iodine in an acidic environment in a range of about 0.3-1.0 ppm. Ammonia may be detected by bromophenol blue (BMP) or bromocresol purple. Carbon monoxide may be detected by molybdenum, tungsten or vanadium color forming agents, palladium, ruthenium or osmium catalysts and reversing agents, such as iron, chromium or cerium, as detailed in U.S. Pat. No. 5,405,583. Hydrogen may be detected by palladium based compounds, titanium dioxide, vanadium oxide, tungsten oxide, molybdenum oxide, yttrium oxide, platinum containing compounds, such as platinum oxides, hydroxides and hydrated oxides and any combination thereof. Nitrogen dioxide may be detected by metallo-porphyrines.

Color pH indicators are widely used and can be utilized, according to some embodiments, by the method of invention, in order to measure the pH of a sample. Thus, the invention includes measurements of presence, as well as amounts, of acids and bases in a given sample.

Thermochromic compounds can be utilized by method of invention for temperature sensing.

According to some embodiments, biomolecules, bacteria and viruses can be detected by vesicle based chromatic filters (Jelinek & Kolusheva, Top. Curr. Chem. DOI 10.1007/1282007, Springer Verlag Berlin, Heidelberg 2007). DNA strands may be detected by using fluorescent molecular beacons. Colorimetric sensing of Nitro aromatic explosives has been demonstrated and can be utilized according to some embodiments in the method of invention (see, e.g., Yingxin Ma et al, Anal. Chem. 2012, 84(19) pp 8415-8421). Sensitive Colorimetric Detection of Warfare Gases by Polydiacetylenes has been demonstrated, e.g., by Jiseok Lee et al., Advanced Functional Materials, Volume 22, Issue 8, pages 1632-1638, April 24, and can be utilized, according to some embodiments, in the method of invention.

In another embodiment the colorimetric sensor is designed to perform Enzyme Linked Immunoassay (ELISA) for specific protein determination and quantification. In such embodiment a 96 element chemochromic array is deposited on the photoactive surface of the image sensor, enabling a colorimetric ELISA sensor.

The colorimetric sensor of invention can be used as a stand alone device or may be embedded (enclosed, incorporated) in various applications in any type of appropriate device like smartphone, laptop computer, tablet or any custom made electronic device with image sensor digital data processing capability.

According to some embodiments, the sensor can be single use or may be reset for multiple us as detailed above. According to some embodiments, means for resetting the sensor are attached to or embedded in the sensor or in the enclosure or device in which the designated sensor is (mounted) embedded.

Other embodiments of the invention include any modifications necessary in the device, which enable it to be used according to embodiments of the invention. According to some embodiments, the sensor or device includes (or is attached to) means for resetting the deposited chemochromic layer, means for radiating/illuminating the chemochromic layer of the sensor from within the device, means for allowing external radiation/light to enter the device in order to illuminate/radiate the chemochromic layer and the like.

According to some embodiments, the sensor of invention can be used as stand alone or be included in a multi sensing custom designed package comprised of a plurality of sensors such that a multiple number of predetermined different analytes, may be tested simultanously or consecutively. According to such embodiments, the plurality of sensors may be arranged in 2D array-linear, rectangular, square, circular or in any appropriate shape or in a 3D shaped arrangement in a customized enclosure or package.

The power required by the sensors can be supplied individually for each sensor from the main electricity, a miniature battery, a solar cell power supply or any equivalent low power source. According to other embodiments, the power may be supplied to the array of sensors from single appropriate power source.

According to some embodiments, the sensor power is supplied by a charging circuit incorporated into the sensor that may be charged remotely by any wireless (Bluetooth, Wi-Fi, NFC, etc.) enabled device, such as a smartphone, computer, tablet etc.

In a particular embodiment the sensor array can be realized in a shape of a wearable bracelet, armband, glue-on patch, any other type of attachable patch (Velcro® etc.) or attached to the fabric of a wearable article and designed for personal monitoring of analytes related to human body, such as alcohol or volatile sulfur compounds (VSC) and volatile organic compounds (VOC) in breath, skin odor or skin hydration monitoring.

In another embodiment the sensor array is designed for simultaneous monitoring of variety of pollutants for environmental control in industry, public areas or home.

In further embodiments, the sensor array is designed for home comfort applications.

In other embodiments, the colorimetric sensors may be combined in an integrated package with other sensors, such as wireless GPS sensors, wireless accelerometers, temperature sensors etc., wherein the package may be designed for specific applications.

The analyte containing sample can be presented to the sensor or sensor array by means, exhaled breath, gas balloon, spray, droplets, a pipette, a syringe a capillary pump or any specifically designed applicator. According to some embodiments, the plurality of sensors may detect any number of analytes. According to some embodiments, each sensor in the array is prepared to detect a different type of analyte. According to some embodiments, any sensor in the array may be prepared such that it may detect more than one analyte.

According to some embodiments, the entire sensor array or each sensor is reset to its original optical mode, using any of the means detailed above. According to further embodiments, each one of the sensors may be reset once used on its own, not depending on the state of use of any of the other sensors in the array.

As noted above, the data file recorded by the sensor, may be transferred by Wi-Fi or Bluetooth, to a device having image analysis software.

The image analysis software should be able to perform spectral analysis, prepare a color histogram, and be able to detect a change in the optical properties of the material, e.g., spectral content, transparency, optical density, etc. Any appropriate image analysis software may be used including algorithms chosen from the following list:

Pearson Correlation

Histogram comparison methods
Correlation
Bhattacharyya distance
Chi-squared Histogram matching distance
Earth Mover's Distance (EMD)
Intersection
Euclidean distance
Manhattan Distance

Phase correlation

Correlation based similarity measures
Sum of Absolute Differences (SAD)
Zero-mean Sum of Absolute Differences (ZSAD)
Locally scaled Sum of Absolute Differences (LSAD)
Sum of Squared Differences (SSD)
Zero-mean Sum of Squared Differences (ZSSD)
Locally scaled Sum of Squared Differences (LSSD)
Normalized Cross Correlation (NCC)
Zero-mean Normalized Cross Correlation (ZNCC)
Sum of Hamming Distances (SHD)
   Many commercial or open source image processing programs are available, such as Xlstat by Addinsoft and ENVI5 by EXELIS.

According to some embodiments, the chemochromic material is incorporated into a layer of transparent inert material, such as polyvinyl alcohol (PVA).

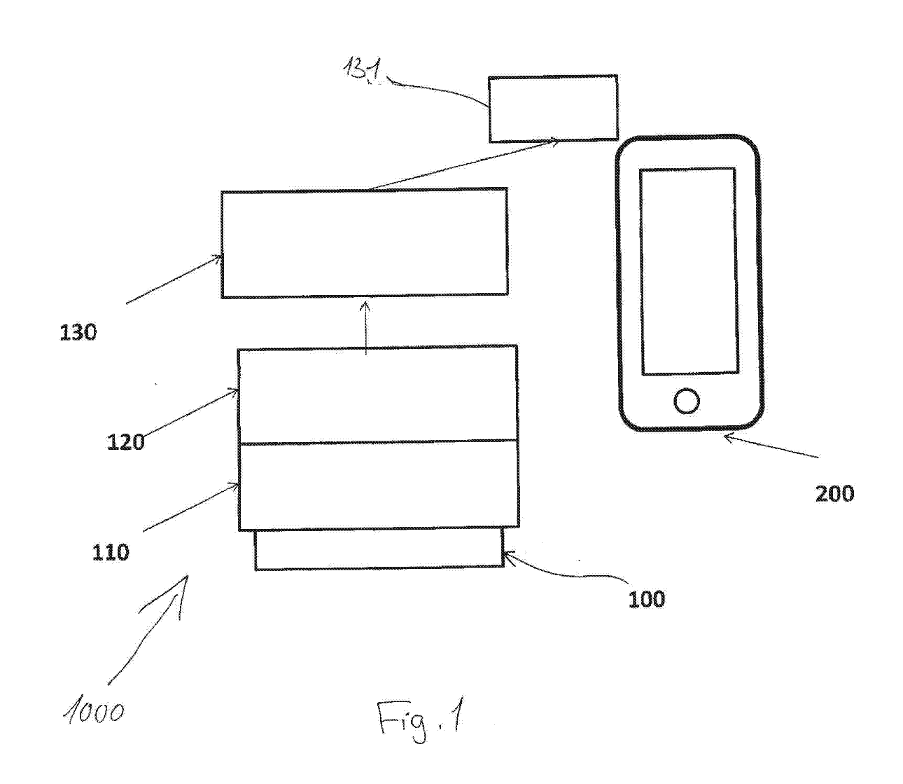

Reference is now made to FIG. 1, presenting a schematic diagram of an embodiment of the chemochromic seonsor (1000) comprising a chemochromic layer (100) coupled to the photoactive surface of a CMOS chip (110), having a microprocessor (120) and being combined with a Wi-Fi/Bluetooth element (130) having antenna (131). The data received from CMOS chip (110) is transmitted via element (130) to smartphone (200).

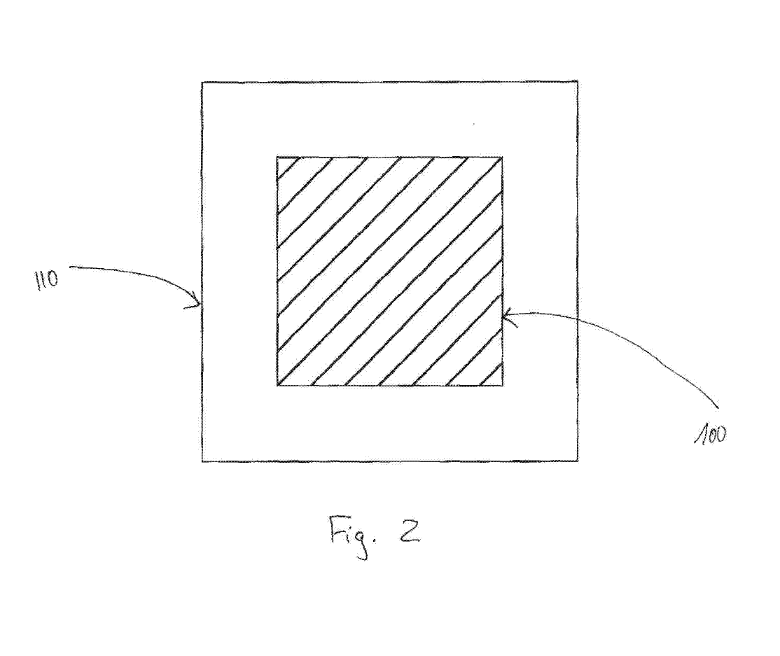

Reference is now made to FIG. 2, presenting a top view of a chemochromic layer (100), found on the photoactive surface of an image sensor (110).

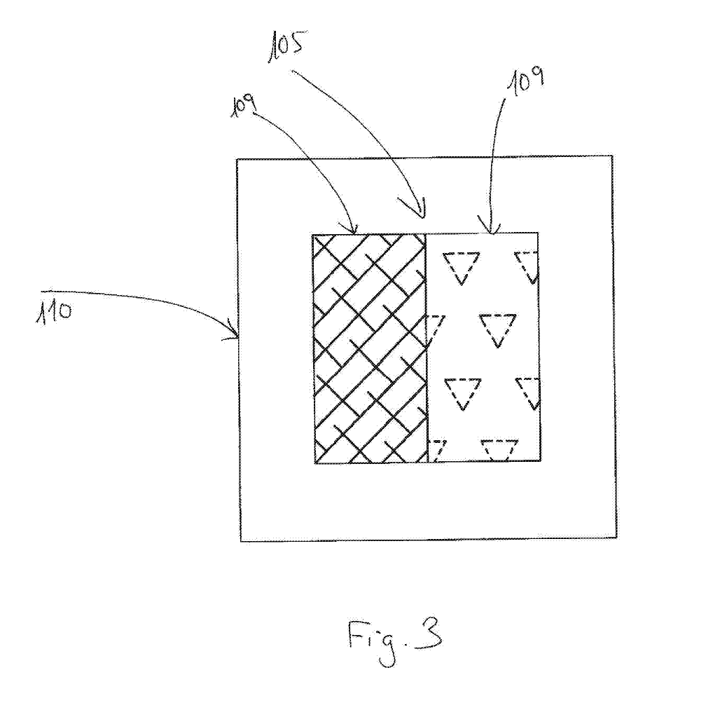

Reference is now made to FIG. 3, presenting a two-element matrix (105), having two different chemochromic regions (107, 109) deposited on the photoactive surface of an image sensor (110). Each one of the chemochromic regions (107, 109) includes a different composition of a chemopchromic material such that each region (107, 109) is designed to detect a different type of analyte.

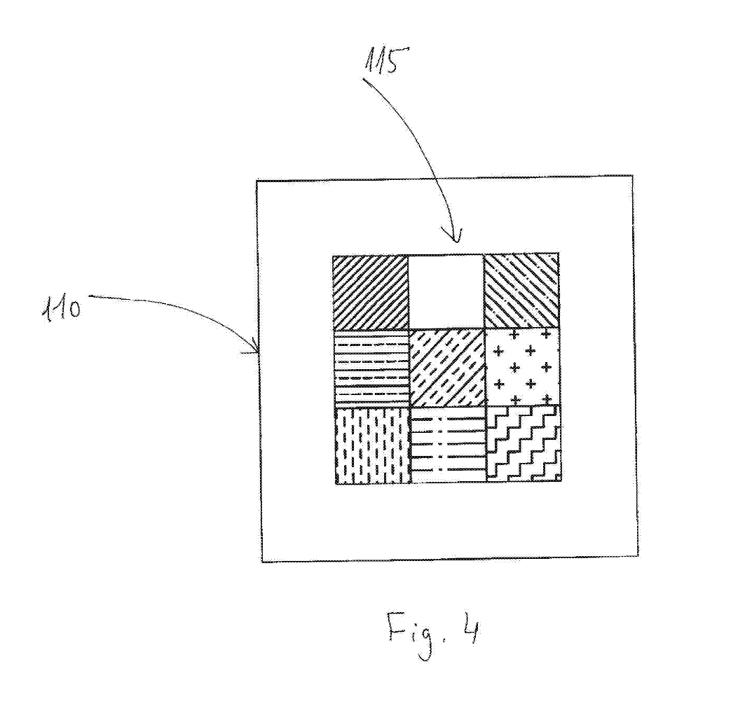

Reference is now made to FIG. 4, presenting a top view of a nine-element matrix of chemochromic regions (115), each for the detection of a different analyte, wherein the chemochromic matrix layer (115) is deposited on the surface of an image sensor (110), such as a CMOS chip.

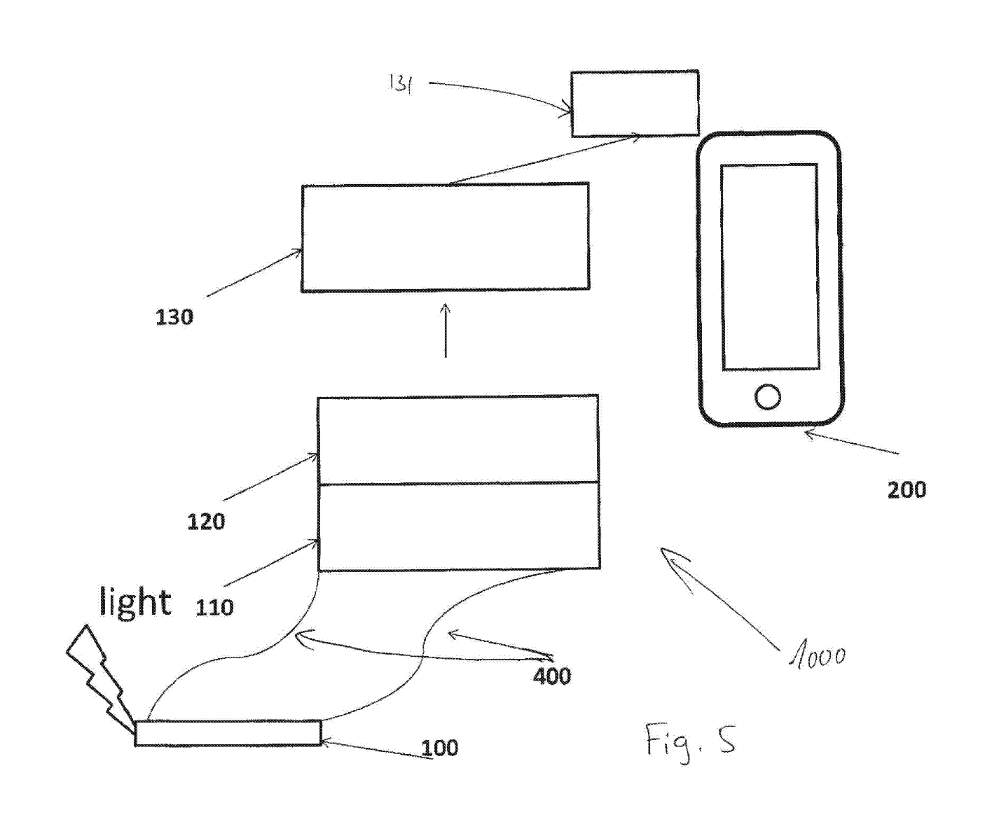

Reference is now made to FIG. 5, presenting an embodiment of a chemochromic sensor (1000) wherein the chemochromic layer (100) is coupled to the photoactive surface of an image sensor (110) by optic couplers (400). Chemochromic layer (100) is exposed to light, the digital optical pattern of light transmitted through the chemochrmoic layer (100) travels through optic coupler (400) to photoactive surface (110) and is recorded by the image sensor's microprocessor (120) and transmitted to smartphone (200) by Wi-Fi/Bluetooth element (130) having antenna (131).

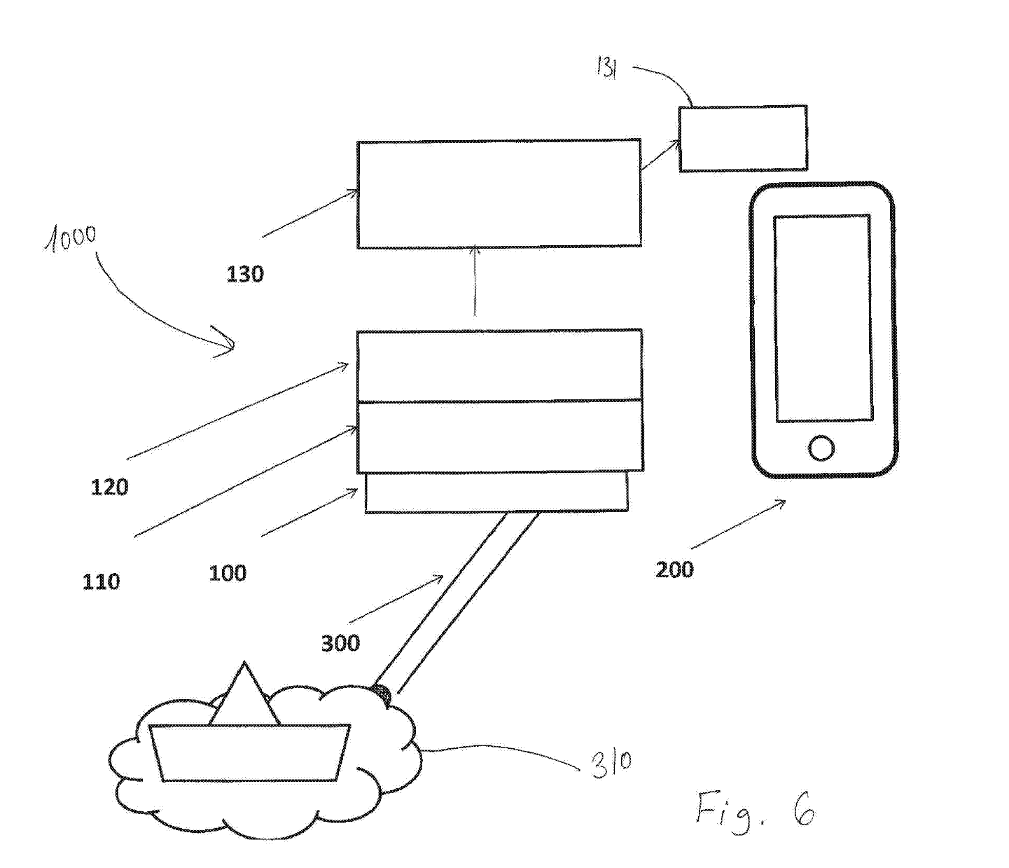

Reference is now made to FIG. 6, presenting an embodiment of chemochromic sensor (1000) wherein liquid analyte (310) is sampled by capillary pump (300), such that capillary pump (300) contacts analyte (310) with chemochromic layer (100) coated on the photoactive surface of an image sensor (110). The chemochromic sensor (1000) further includes microprocessor (120), Wi-Fi/Bluetooth chip (130) with antenna (131), which transfer recorded digital optical patterns of light transmitted through the chemochromic layer (100) to smartphone (200).

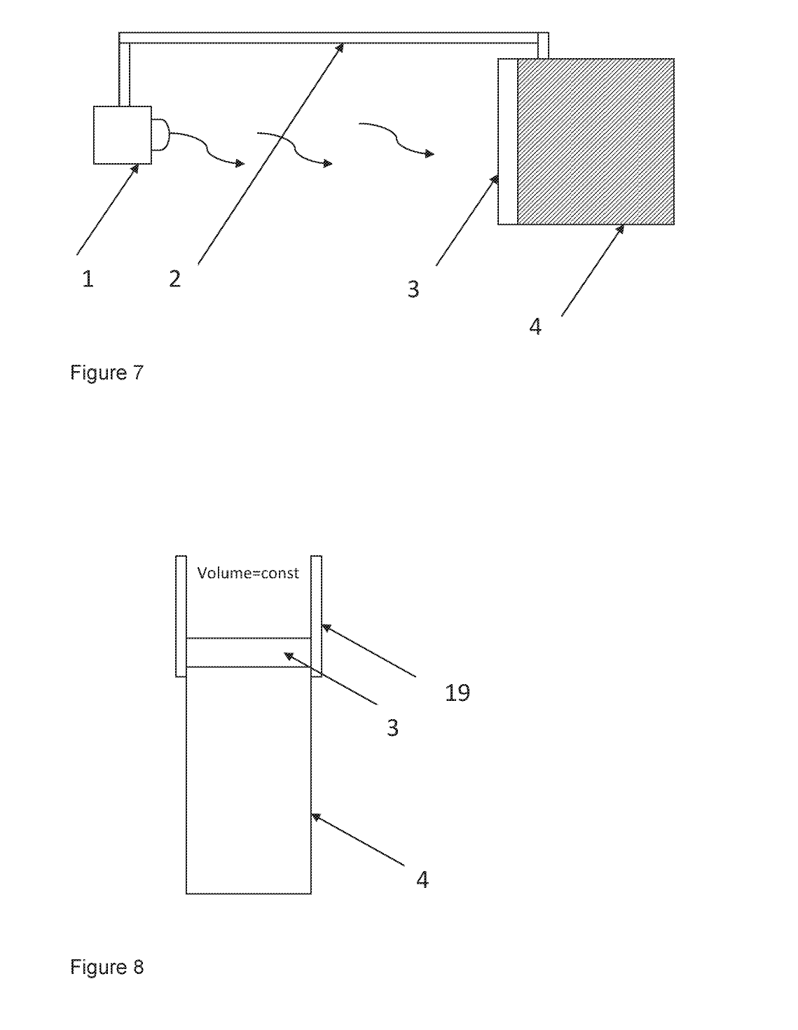

Reference is now made to FIG. 7, presenting an embodiment of the invention according to which UV source (1) is attached by radiation source holder (2) to image sensor (4), wherein chemochromic layer (3) is coated on the photoactive surface of image sensor (4). The use of such an embodiment is beneficial, e.g., as detailed in Example 8, for the detection of DNA, where the UV source (1) is used for DNA fluorescence excitation.

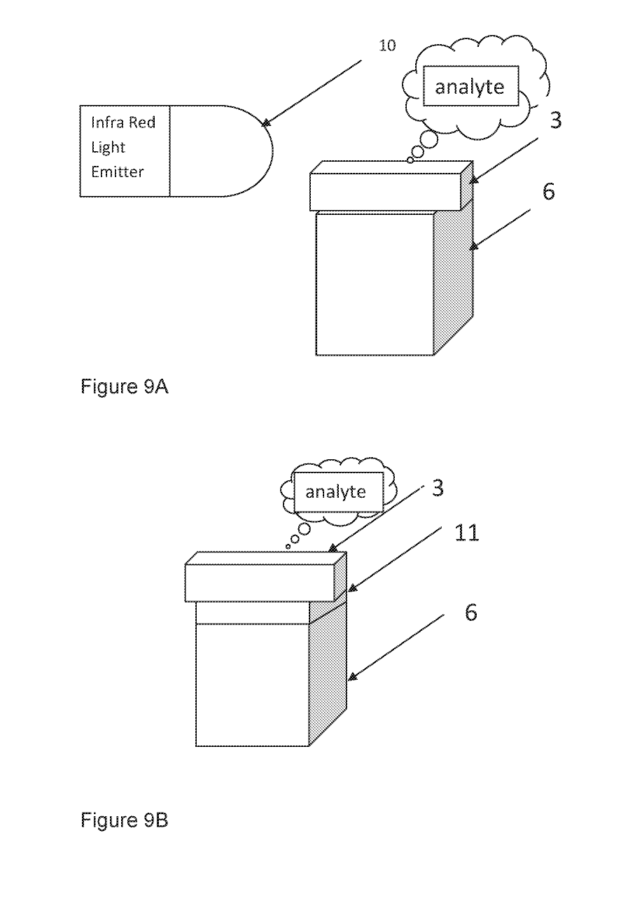

Reference is now made to FIG. 8, presenting an embodiment of a constant volume sampling reservoir (19) that is placed on (or somehow attached to) chemochromic layer (3), which is placed on image sensor (4). Such a volume sampling reservoir (19) may be used in order to expose chemochromic layer (3) to a constant volume of a sample (not shown).

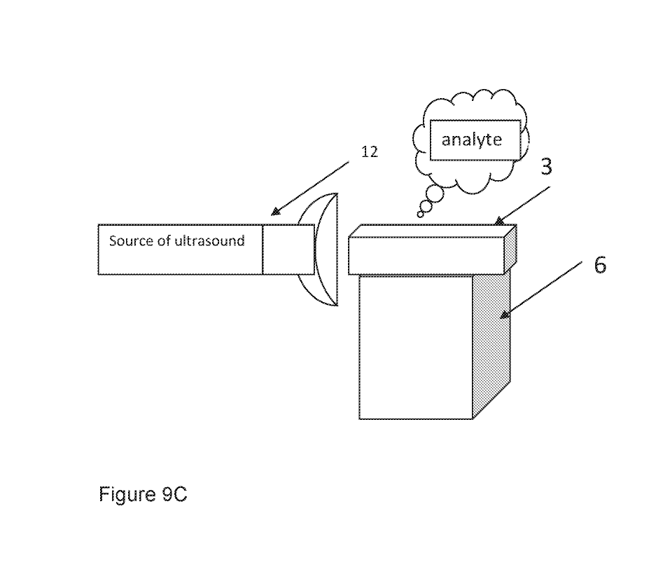

Reference is now made to FIG. 9A, presenting chemochromic layer (3) that is applied onto the photoactive surface of image sensor (6). Chemochromic layer (3) is exposed to an analyte, which changes the optical state of the chemochromic material included in the chemochromic layer (3). After exposure (and measurement of the chemochromic changes occurring as a result of the exposure) the chemochromic layer (3) is returned to its original optical state by IR radation source (10).

Reference is now made to FIG. 9B, presenting a chemochromic layer (3) that is applied to image sensor (6). Chemochromic layer (3) is exposed to an analyte, which changes the optical state of the chemochromic material included in the chemochromic layer (3). After exposure (and measurement of the chemochromic changes occurring as a result of the exposure) the chemochromic layer (3) is returned to its original optical state by heating element (11).

Reference is now made to FIG. 9C, presenting a chemochromic layer (3) that is applied to image sensor (6). Chemochromic layer (3) is exposed to an analyte, which changes the optical state of the chemochromic material included in the chemochromic layer (3). After exposure (and measurement of the chemochromic changes occurring as a result of the exposure) the chemochromic layer (3) is returned to its original optical state by ultrasound source (12).

It is emphasized that in the embodiments of this invention the image sensor is utilized as a counter of the colors integrated on the photoactive surface, wherein the specific color of each pixel on the photoactive surface has no importance.

### Example 1

### Mercaptan Detection

A colorimetric sensor designed for the detection of volatile sulfur compounds present in human breath was prepared by attaching the chemochromic layer the photoactive surface of a bluetooth enabled image sensor (SONYICX205AL coupled to a Broadcom BCM 2046 Single-Chip Bluetooth Mono Headset IC and powered by a solar cell (Applied Materials) connected to a microcontroller for solar cell (F28M35x Texas Instrument)). The chemochromic layer comprised 1.5 mg/cm2 of CuSO4 incorporated into a 70 micron layer of polyvinyl alcohol (PVA) and cast onto a thin backing (coupler) of PET film. A series of measurements is performed with a number of such (identical) chemochromic layers deposited on the photoactive surface of the image sensor, such that a new layer is placed on the sensor for each one of the experiments. Reference data from a non-exposed sensor is recorded on an i-phone and is followed by a series of measurements with exposure to a constant volume of 100 ml air sample with different concentration of mercaptane, also recorded. Each measurement is performed after the replacement of the chemochromic layer with a new layer. The initial reference data is compared to data files obtained from exposed chemochromic layers using image analysis software, thus measuring the optical difference caused by the exposure of the chemochromic material to the mercaptan. The expected results of the above tests are in the ranges of 1-6 ppm mercaptan and 0-1 ppm mercaptan, respectively.

### Example 2

### Alcohol Detection

A colorimetric sensor, as detailed in Example 1, was designed for the detection of alcohol present in human breath. The chemochromic layer comprised 30% polyvinyl alcohol, 0.1% CR-546 (N,N-dioctylamino-4′-trifluoroacetyl-2′-nitroazobenzene) Fluka Cat. No 08709 (Selectophore™) and 69.9% water on a polyethylene phthalate (PET) inert backing having a thickness of 100 microns. A series of measurements is performed with a number such chemochromic layers deposited on the image sensor. Reference data from a non-exposed sensor is recorded on an i-phone and is followed by a series of measurements with exposure to a constant volume of 100 ml air sample with different concentration of ethyl alcohol, also recorded. Each measurement is performed after replacement of the chemochromic layer with a new unexposed layer. The initial reference data is compared to data files obtained from exposed chemochromic layers using image analysis software, thus measuring the optical difference caused by the exposure of the chemochromic material to alcohol. The expected relative optical absorption is from the blue to rose spectral change due to alcohol concentrations in the range of 0-50 ppm.

### Example 3

### Protein Detection

A colorimetric sensor, as detailed in Example 1, was designed for the detection of proteins, comprises the chemochromic material Coomassie Brilliant Blue G-250 such that the active region of each filter had 0.5 mg/cm2 of Coomassie incorporated into a 50 micron layer of polyvinyl alcohol (PVA) cast onto a transparent polyethylene terephthalate (PET) backing. The protein selected for this example consists of Bovine Serum Albumin (BSA) water based solution. A series of measurements is performed with a number such chemochromic layers deposited on the image sensor. Reference data from a non-exposed sensor is recorded on an i-phone and was followed by a series of measurements with exposure to a constant volume of 5 microliters of water solution placed on the surface of the chemochromic layer, also recorded. Each measurement is performed after replacement of the chemochromic layer with a new unexposed layer. The initial reference data is compared to data files obtained from exposed chemochromic layers using image analysis software, thus measuring the optical difference caused by the exposure of the chemochromic material to varying concentrations of BSA in the solution. The expected results of the above tests in ranges of 100-1000 ng BSA and 1 μg-1 mg BSA respectively.

### Example 4

### Acetone Detection

A colorimetric sensor, as detailed in Example 1, was designed for the detection of acetone, comprised a chemochromic layer composed of chemochromic material Sodium Nitroprusside combined with NaOH in composition of 0.7 mg/cm2 of sodium Nitroprusside and 0.01 mg/cm2 of NaOH incorporated into a 70 micron layer of polyvinyl alcohol (PVA) cast onto a transparent polyethyleneterephthalate (PET) backing. Reference data from a non-exposed sensor is recorded on an i-phone and was followed by a series of measurements with exposure to a constant volume sample of 300 ml of acetone vapors in air, also recorded. Each measurement is performed after replacement of the chemochromic layer with a new unexposed layer. The initial reference data is compared to data files obtained from exposed chemochromic layers using image analysis software, thus measuring the optical difference caused by the exposure of the chemochromic material to varying concentrations of acetone vapor in the sample. The expected results of the above tests are in ranges of 100-600 ppb and 600-2000 ppb acetone, respectively.

### Example 5

### pH Detection

A colorimetric sensor, as detailed in Example 1, was designed for the detection of acetone, comprised a chemochromic layer composed of 0.7 mg/cm² of phenolphthalein incorporated into a 70 micron layer of polyvinyl alcohol (PVA) cast onto a transparent polyethyleneterephthalate (PET) backing. Reference data from a non-exposed sensor is recorded on an i-phone and is followed by a series of measurements with exposure of the surface of the chemochromic layer to a sample of 1 μL solution of NaOH in water, also recorded. Each measurement is performed after replacement of the chemochromic layer with a new unexposed layer. The above measurements is performed using known concentrations of the NaOH base. The expected results of the above tests are in the ranges of 7<pH<14.

### Example 6

### Free Chlorine Detection

A colorimetric sensor, as detailed in Example 1, comprised a chemochromic layer composed of the chemochromic material DPD (N,N-diethyl-p-phenylene-diamine) at concentration 0.1 mg/cm2 of DPD incorporated into a 20 micron layer of polyvinyl alcohol (PVA) cast onto a transparent polyethylene terephthalate (PET) backing. The chemochromic layers were attached to the photoactive surface of the image sensor. Reference data from a non-exposed sensor is recorded on an i-phone and followed by a series of measurements with exposure of the surface of the chemochromic layer to a sample of 1 μL solution of chlorine in water, also recorded. Each measurement is performed after replacement of the chemochromic layer with a new unexposed layer. The above tests are performed using known concentrations of free chlorine, The expected results of the above tests are in ranges of 10-1000 ng chlorine and 1 μg-1 mg chlorine.

### Example 7

### Aliphatic Amines Detection

A colorimetric sensor, as detailed in Example 1, comprising a chemochromic layer composed of the chemochromic material 1-chloro-2:4-dinitrobenzene (CDNB), was prepared so that the active region of each filter had 0.2 mg/cm2 of CDNB incorporated into a 50 micron layer of polyvinyl alcohol (PVA) cast onto a transparent polyethylene terephthalate (PET) backing. The above chemochromic layers were attached to the photoactive surface of the image sensor. Reference data from a non-exposed sensor is recorded on an i-phone and is followed by a series of measurements with exposure of the surface of the chemochromic layer to 3 μL of aliphatic amine (AA) based water solution, also recorded. The data files obtained from the above measurements are wirelessly transferred to the i-Phone and are processed by an image processing software. The above tests are performed using known concentrations of AA, The expected results of the above tests are in concentration ranges of 10-1000 ng AA and 1 μg-1 mg AA.

### Example 8

### DNA Detection

A UV source (1) is used to illuminate the chemochromic layer of the sensor. This embodiment exemplifies the detection of DNA, where a UV source is used for DNA fluorescence excitation as detailed in this example.

Colorimetric sensors, as detailed in Example 1, having a fluorescent chemochromic layer for DNA detection, comprising the chemochromic material ethydium bromide (EB) composed 0.7 mg/cm2 of EB incorporated into a 70 micron layer of polyvinyl alcohol (PVA) deposited onto a transparent polyethyleneterephthalate (PET) backing. Molecules of EB are known to adhere to DNA strands and fluoresce under UV light. Each such chemochromic layer is attached to the photoactive surface of the CMOS sensor, as detailed in Example 1.

Reference data from a non-exposed sensor is recorded on an i-phone. Additional tests are performed using known concentrations of DNA in water solution (Lambda HIND DNA from BioRad) of 10 μL volume deposited on the surface of the above designed chemochromic layers. For DNA fluorescence excitation, a UV lamp (358 nm) is used. The data files obtained in these measurements are wirelessly transferred to the i-Phone and processed by an image processing software. The expected results of the above tests are in ranges of 10-1000 ng DNA.

### Example 9

### Lead Detection

Colorimetric sensors of invention for detection of concentrations of Lead Acetate in water are prepared by depositing a lead sensitive chemochromic layer comprising the chemochromic material sodium sulfide of 0.5 mg/cm2 of sodium sulfide incorporated into a 50 micron layer of polyvinyl alcohol (PVA) cast onto a transparent polyethylene terephthalate (PET) backing, onto the photoactive surface of the CMOS sensor, as detailed in Example 1. Reference data from a non-exposed sensor is recorded on an i-phone and is followed by a series of measurements with exposure of the surface of the chemochromic layers to different concentrations of lead in a sample of 100 μL, placed on the sensor in liquid form, also recorded. The data files obtained in these measurements are wirelessly transferred to the i-Phone and processed by an image processing software. The concentration is expected to be in the range of 1 nM-1 mM.

### Example 10

### ELISA Colorimetric Sensor

A colorimetric sensor based on components, as detailed in Example 1, was designed to perform Enzyme Linked Immuno Assay (ELISA) for sensitive detection of specific proteins (Albumin). A 100 element array of chemochromic material segments was deposited on the photoactive surface of a 106 pixels CMOS image sensor, each segment spanning 100×100 pixels. Each of the segments (wells) contains a specific concentration of Rabbit anti-Equine IgG, IgM, IgA Secondary Antibody, labeled with fluorescent Rhodamin (Pierce) and polymerized in a polyacrylamide layer (chemical polymerization, 4% AA+TEMED+APS). The concentration range of the labeled antibody is 10-1000 pg/segment (well). The CMOS image sensor with the polymerized chemochromic segments is incorporated in a 2 microliter volume vessel. This reservoir with sensor is mounted with a UV-emitting LED (without plastic optics on pin structure) in an opaque enclosure equipped with a capillary for test sample delivery. After delivery of the albumin protein probe, the chemochromic array is illuminated with the LED emitting UV light and the fluorescent light pattern was recorded by the image sensor, transmitted to an i-phone by Bluetooth, and processed by a software application intended for fluorescent light analysis from each segment/well. The achieved level of detection is expected to be about 10 pg/well.

### Example 11

### Skin Hydration Sensor Armband

A colorimetric sensor, based on components as detailed in Example 1, was designed for skin hydration sensing. A two-element chemochromic layer was deposited on the photoactive surface of the image sensor comprising the chemochromic materials CoCl2 for monitoring the level of water in sweat and a crown ether chelate complex 15-Crown-5 (to monitor the concentration of sodium ions in sweat. The sensor is mounted on a flexible plastic band in the form of an armband. A data processing software was developed to simultaneously monitor the level of both analytes and their ratio.

Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

We claim:

1. A wireless colorimetric sensor for detecting, quantifying, or both detecting and quantifying, at least one analyte in a sample, said sensor comprising a light transparent chemochromic layer applied onto the photoactive surface of an image sensor, or attached thereto via an optic coupler, wherein said chemochromic layer comprises a chemochromic material that is optically altered when contacted with said analyte.

2. The wireless colorimetric sensor according to claim 1, wherein the image sensor is a CMOS or CCD sensor.

3. The wireless colorimetric sensor according to claim 1, wherein the chemochromic material is uniformely dispersed in a transparent media.

4. The wireless colorimetric sensor according to claim 1, wherein the chemochromic layer covers only part of the photoactive surface of the image sensor.

5. The wireless colorimetric sensor according to claim 1, wherein the chemochromic layer covers the whole photoactive surface of the image sensor.

6. The wireless colorimetric sensor according to claim 1, wherein the chemochromic layer includes more than one chemochromic material.

7. The wireless colorimetric sensor according to claim 1, wherein the chemochromic layer includes a matrix of subsections, wherein each subsection comprises a different chemochromic material.

8. The wireless colorimetric sensor according to claim 1, wherein a part of the chemochromic layer does not include a chemochromic material.

9. The wireless colorimetric sensor according to claim 1, wherein more than one chemochromic layers are attached to the photoactive surface of the image sensor via more than one optical couplers.

10. The wireless colorimetric sensor according to claim 1, wherein the optical coupler is a flexible light guide, an optical fiber or a bundle of optical fibers.

11. The wireless colorimetric sensor according to claim 1, which is disposable.

12. The wireless colorimetric sensor according to claim 1, which is for multiple use.

13. The wireless colorimetric sensor according to claim 1, wherein the chemochromic material is reset after exposure to the analyte.

14. A method for detecting at least one analyte with a colorimetric sensor comprising an image sensor, wherein a chemochromic layer comprising a chemochromic material is applied onto the photoactive surface of said image sensor, or attached thereto via an optic coupler, said method comprising:
exposing at least part of the chemochromic material to an analyte;
transmitting light through at least part of the chemochromic layer;
recording a digital optical pattern of light transmitted through at least part of the chemochromic layer;
transmitting said digital optical pattern to an image processing device; and
comparing said digital optical pattern with a digital optical reference pattern.

15. The method according to claim 14, wherein the digital optical pattern is transmitted to the image processing device by Wi-Fi, Bluetooth or near field communication.

16. The method according to claim 14, wherein the digital optical reference pattern is obtained by:
transmitting light through at least part of the chemochromic layer before is it exposed to the analyte;
transmitting light through at least part of the chemochromic layer of any appropriate sensor; or
transmitting light through regions of the chemochromic layer that do not contain chemochromic material.

17. The method according to claim 14, wherein the digital optical reference pattern includes quantitative data.

18. The method according to claim 14, wherein the chemochromic layer includes more than one chemochromic material for detecting more than one analyte.

19. The method according to claim 14, wherein each optic coupler is positioned such that a distal end thereof, on which the chemochromic layer is found, is placed in a region appropriate for identifying each analyte.

20. The method according to claim 14, wherein at least part of the chemochromic material is exposed to the analyte by spreading a sample containing the analyte on the chemochromic layer, by a capillary pump dipped in a vessel containing the analyte, a spray, aerosol, gas stream or brush.
